(19) United States
(12) Patent Application Publication
(10) Pub. No.: US 2004/0136888 A1
(43) Pub. Date: Jul. 15, 2004

(54) Incense Burner

(76) Inventors: Satoshi Shimizu, Akashi-shi (JP); Takao Kanba, Kobe-shi (JP); Haruo Ishikawa, Kobe-shi (JP)

Correspondence Address: SHERIDAN ROSS PC, 1560 BROADWAY, SUITE 1200, DENVER, CO, 80202

(21) Appl. No.: 10/625,948

(22) Filed: Jul. 23, 2003

(30) Foreign Application Priority Data
Jul. 29, 2002 (JP) PAT. 2002-219351
Jul. 30, 2002 (JP) PAT. 2002-221003

(51) Int. Cl. A61L 9/00

(52) U.S. Cl. 422/305; 422/306; 422/307

(57) ABSTRACT

The present invention provides an incense burner which effectively prevents the user from an easy touch on its high temperature parts, can be used at a plurality of temperature ranges, and can let the user enjoy comfortably light art and aroma for a long period of time with ease. The incense burner of the present invention has a container for accommodating the material to be heated, a heating plate provided beneath the container, a heater for heating the heating plate and a supporting component for supporting the heating plate.

### BACKGROUND OF THE INVENTION

The present invention relates to an incense burner provided with a heat source for heating the material to be heated which emits aroma to the atmosphere.

In the past, there have been a number of such incense burners which employ an electric bulb or a candle as their heat sources and use porcelain as their exterior enclosure of its body. There is also known such an incense burner having a function of performing light-art in addition to that of emitting aroma.

The known type of the incense burner that employs the electric bulb or candle is designed to elevate the temperature of the atmosphere inside the exterior enclosure and to heat the material to be heated at the temperature of the atmosphere in the enclosure, and thus has a disadvantageous problem that the temperature of the enclosure undesirably rises when the temperature of the atmosphere inside the exterior enclosure rises. There has also been another problem that the user of the incense burner unintentionally touches a high temperature part of the enclosure, because it uses the light bulb or candle as its light source.

For causing the dried leaves of green tea or black tea to emit an aroma component, a high temperature around 200° C. is required, whereas for heating an aroma oil to emit its aroma, it is desirable to use a moderate temperature not exceeding about 70° C. at which water does not evaporate immediately because the aroma oil is used while it is dropped into water.

Heretofore, there has no such incense burner that can be used comfortably for the both purposes. The reason for this is that the temperature of the atmosphere in the exterior enclosure of the incense burner which uses the electric bulb or candle cannot be varied greatly by adjusting the consumption amount of the wax of the candle or the electric power for the bulb. There has also been such a bulb-type incense burner that can adjust the temperature of the heating part by varying the distance between the bulb and the heating part, but there is a limit for setting the desired temperature difference.

Moreover, there is another problem that the exchange of the electric bulb is necessary and thus the use for a long time is not possible. Although the incense burner which uses the candle as its light source is preferred to that which uses the electric bulb for its visual effect, it has a disadvantage of unexpectedly causing a fire in the use for a long period of time.

The present invention is proposed for solving the above-mentioned problems and it is an object of the present invention to provide an incense burner that can be used safely for a long period of time.

Another object of the present invention is to provide an incense burner that can heat the leaves of green tea or black tea at a temperature sufficient for causing them to emit an aroma component while suppressing the temperature rise of the exterior enclosure.

It is another object of the present invention to provide an incense burner that prevents an easy touch of the user at its part of high temperature.

It is further another object of the present invention to provide an incense burner that can comfortably be used at a plurality of temperature ranges.

It is still another object of the present invention to provide an incense burner that makes the exchange of the light source unnecessary, prevents a temperature of the light source from being high, and makes it possible to enjoy the light art and aroma for a long time, comfortably at ease.

It is still further another object of the present invention to provide a low cost incense burner that can be easily assembled.

### SUMMARY OF THE INVENTION

In order to solve the above-mentioned problems, the incense burner in accordance with the present invention comprises a container for accommodating material to be heated; a heating plate disposed beneath the container; a heater for heating the heating plate; and a supporting member for supporting the heating plate. The present invention has a function of realizing the incense burner that can be used safely and continuously for a long period of time.

Although the novel features of the present invention are no more than those particularly described in the appended claims, it is to be noted that the present invention, both as to construction and content, will be better understood and appreciated, along with other objects and features thereof, from the following detailed description taken in conjunction with the appended drawings.

### BRIEF DESCRITION OF THE DRAWINGS

FIG. 1 is a cross-sectional view of the essential configuration of the incense burner in Embodiment 1.

FIG. 2 is an (enlarged) cross-sectional view of a part around the heater of Embodiment 1 of the present invention.

FIG. 3 is a cross-sectional view of a part around a rubber ring of Embodiment 1 of the present invention.

FIG. 4 is a bottom view of the heating body unit in Embodiment 1 of the present invention while it is being assembled.

FIG. 5 is a bottom view of the upper frame unit in Embodiment 1 of the present invention.

FIG. 6 is a part oblique view of the essential components for showing a state of attachment of a pillar to the upper frame unit.

FIG. 7 is a part cross-sectional view of the essential components for showing the state of attachment of a pillar to the upper frame unit.

FIG. 8 shows a circuit diagram of the control part in Embodiment 1.

FIG. 9 is a view showing a state at which the incense burner of the present invention falls down.

FIG. 10 is a view showing a state at which the incense burner of the present invention is placed on an uneven installation plane.

FIG. 11 is a cross-sectional view of the essential configuration of the incense burner in Embodiment 2 of the present invention.

FIG. 12 is a cross-sectional view of the essential configuration of the incense burner in Embodiment 3 of the present invention.

FIG. 13 is a cross-sectional view of the essential configuration of the incense burner in Embodiment 4 of the present invention.

FIG. 14 is a bottom view of the container in Embodiment 4 of the present invention.

It will be recognized that some or all of the Figures are schematic representations for the purpose of illustration, and do not necessarily depict the actual relative sizes or locations of the elements shown.

### DETAILED DESCRIPTION OF THE INVENTION

An incense burner from an aspect of the present invention comprises: a container for accommodating material to be heated; a heating plate disposed beneath the container; a heater for heating the heating plate; and a supporting member for supporting the heating plate. The container is mounted on the heating plate so that its bottom face may be in substantially close contact with the heating plate, and preferably, the heater, the heating plate and the container are all in substantial contact with each other. Since the material to be heated can be heated by the heater without placing any air layer between them in the stated configuration, it is possible to provide such an incense burner that can heat the leaves of green tea or black tea to a temperature sufficient for causing them to emit aroma components therefrom.

In the above-described incense burner from another aspect of the present invention the container includes a set of a first container and a second container which have different shapes and are interchangeably mountable on the heating plate; the first container is so disposed that its bottom being in substantial contact with the heating plate; and the second container is so disposed that its bottom being spaced apart from the heating plate. The present invention makes it possible to lower the temperature inside the second container when it is used, as compared with the temperature inside the first container when it is used and thus to provide an incense burner that can be comfortably used at a plurality of temperature ranges.

For instance, in a case of causing the green tea or black tea leaves to emit their aroma component, they are heated at a high temperature (around 200° C.) by using the first container that realizes a high heat conductivity. In contrast, in a case of heating an aroma oil for example, it is heated at a low temperature (not higher than about 70° C.) by using the second container whose heat conductivity is worse than that of the first container.

In the above-mentioned incense burner from another aspect of the present invention, the second container is mounted on a top cover which covers a periphery of the heating plate. By configuring as stated, it is possible to provide a gap between the second container and the heating plate in a simple configuration. Since this gap suppresses the calorific value of heat which is conducted from the heating plate to the second container, it is possible to lower the temperature inside the second container.

In the above-described incense burner from another aspect of the present invention, the second container has at least one leg for providing a space between the heating plate and the bottom of the second container. With this configuration, it is possible to make a thermal resistance between the second container and the heating plate larger (the total cross-sectional areas of the legs are far less smaller than the cross-sectional area of the second container, and the calorific value conducted through the legs is small.). Since the legs moderately suppress the calorific value conducted from the heating plate to the second container, it is possible to lower the temperature inside the second container. Since air flowing among the legs of the second container robs the heating plate of the unnecessary heat, it is possible to suppress the temperature rise of the outer enclosure of the incense burner. By selectively using the first container and the second container made of different materials which are different in their thermal conductivity, it is possible to provide the incense burner that can be comfortably used at a plurality of temperature ranges.

The above-mentioned incense burner from another aspect of the present invention employs the heating plate and the bottom of the first container which are both made of metal. With this configuration, the thermal resistance of a path from the heater to the first container through the heating plate is made small, and the heat generated by the heater is effectively conducted to the first container. Therefore, it is possible to heat the green tea or black tea leaves up to a temperature sufficient for causing them to emit their aroma component.

In the above-described incense burner from another aspect of the present invention, the bottom of the second container is made of ceramic, porcelain or steatite. The thermal conductivity of the second container is a value suitable for heating the aroma oil (the thermal conductivity is not so large as metals, and not so small as to shut the heat). With this configuration, it can suitably heat the aroma oil. Since the heat generated by the heater is suitably conducted to the second container, it is possible to suppress the temperature rise of the outer enclosure. By selectively using the first container and the second container made of different materials which are different in their thermal conductivity, it is possible to use the incense burner at a plurality of temperature ranges.

In the above-mentioned incense burner from another aspect of the present invention, the second container has a handle part along its peripheral part, and at least one leg on its bottom part for providing a space between the top cover and the bottom of the second container, and is retained on the top cover with the leg. With this configuration, it is possible to lower the temperature of the second container, and to cause the top cover to hold the second container in a case where the main body of the incense burner inclines or moves.

In the above-described incense burner from another aspect of the present invention, the second container has a handle part along its peripheral part, and at least one leg on its bottom for providing a space between the top cover and the bottom of the second container, and the leg is provided in a direction of the handle part. With this configuration, the air heated by the heating plate is interrupted by the leg of the second container, and thus it is possible to prevent the temperature rise at the handle part.

In the above-mentioned incense burner from another aspect of the present invention, the heater is urged to a direction of the heating plate by a top cover which covers a peripheral part of the heating plate, a heater spring, and a heater cover on which one end of the heater spring rests; and the incense burner comprises a first conduction plate provided between the heating plate and the heater; and a fixing plate provided on the heater cover and positioned by the top cover or the heater cover, wherein the first conduction plate and the heater are positioned by the fixing plate. By doing so, the configuration of the incense burner is simplified, and it is enabled to insulate the conduction heat from the first conduction plate and the heater.

The above-described incense burner from another aspect of the present invention, further comprises a rubber ring provided between the heating plate and the top cover, wherein the rubber ring is covered by the top cover, thereby to provide a space between the heating plate-side tip end of the top cover and the heating plate. With this configuration, it is possible to seal at a part where the temperature falls as much as that corresponds to the space above the heating plate, thereby to prevent the water or the aroma oil from intruding into the interior of the main body.

In the above incense burner from another aspect of the present invention, the heater is urged to a direction of the heating plate by the top cover which covers a periphery of the heating plate, a heater spring and a heater cover on which one end of the heater spring rests, and the heater cover has a bearing part for supporting the heating plate. With this configuration, even if the heating plate is pushed from the outside to the direction for the heater with more force than force derived from the heater spring for urging, the bearing part of the heater cover can endure the exertion of the force, and thus it is possible to prevent the heater from being exerted an excessive load.

The above-described incense burner from another aspect of the present invention, further comprises: a rubber ring provided between the heating plate and the top cover which covers a periphery of the heating plate, thereby to provide a gap between the bearing part of the heater cover on which one end of the heater spring rests and the heating plate, wherein the gap does not exceed a compression margin of the rubber ring. With this configuration, even if the heating plate is pushed against the heater, the heating plate is stopped by hitting on the bearing part of the heater cover. Even in a case wherein the heating plate moves to its maximum extent, the gap between the top cover and heating plate can maintain its state of being sealed by the rubber ring, because the displacement of the heating plate is not greater than the compression margin of the rubber ring.

The above-described incense burner from another aspect of the present invention, further comprises a second conduction plate provided between the heater spring and the heater, wherein the second conduction plate is positioned by the fixing plate. With this configuration, it is possible to suppress the temperature rise of the fixing plate and to improve the assembling property.

In the above-mentioned incense burner from another aspect of the present invention, the heater cover is retained on the top cover by rotating it. With this configuration, the heater spring is prevented from its coming off, thereby to make the assembling operation easy.

An incense burner in accordance with another aspect of the present invention comprises: a heater; a heating plate; a top cover having a vertically piercing opening which has an inner fringe part provided along a periphery of the opening for holding the heating plate so that it may not escape upwards, and a lateral part for holding the heating plate and the heater stored in the opening from a bottom and for regulating positions of these components directly or by the aids of other components so that they may not move in a lateral direction or rotate; a heater spring for urging the heater to a direction of the heating plate; and a heater cover fixed on an underside of the top cover for providing an approximately closed space defined between the top cover and the heater cover, which accommodates at least the heater spring, the heater and the heating plate, wherein the heater cover supports the heater spring upwards.

By accommodating the heater in the approximately closed space, it is possible to prevent the water or aroma oil from intruding into the heater from the outside.

Since only the heater spring substantially urges the heater, the heater is in close contact with the heating plate (no force works to compete with the force of the heater spring, for instance, no lateral force is exerted on the heater), thereby to give a high thermal efficiency. Since no undue force is exerted on the heater, there is no fear of breaking the heater.

Preferably, a structure is taken, wherein the top cover can be engaged with the heater cover by rotating the heater cover. By doing so, it is possible to reduce the step required for the assembling operation. Since the heating plate and the heater are laterally (in a direction perpendicular to the axis of rotation of the top cover) position-regulated in the opening of the top cover, when the top cover is rotated, they rotate together with the top cover as a single body. Since the heating plate and the heater are mounted on the heater cover through the heater spring, a sliding is created between the heater cover and the heater spring and/or between the heater and the heater spring when the top cover is rotated. At the time of rotation, no undue force is exerted on the respective members. After the engagement of the top cover with the heater cover, any member does not move.

The first conduction plate may be inserted between the heating plate, and/or the second conduction plate may be inserted between the heater and the heater spring. In order that none of these members, accommodated in the opening of the top cover from its underside, may move laterally or rotate, the peripheral part of the top cover regulates positions of these members directly or through other members.

An assembling operator makes the heater cover accommodate the heating plate, the heater and the like, and engage them with the top cover by rotating the heater cover. The assembling operator can assemble all of these members at once, in an operation approximately equivalent to an operation that is required for assembling only two members (heater cover and the top cover).

In a state where the heater cover and the top cover are assembled, the heater and the first conduction plate and/or the second conduction plate are pushed against the heating plate only by the force of the heater spring. It is to be noted that except for the force of the heater spring, these members are position-regulated only by the lateral part of the opening of the top cover, and no force is exerted thereon for fixing them. Any force for competing with the force of the heater spring does not work. By virtue of the heater spring, the heater is in close contact with the heating plate, directly or through another member, thereby to give a high thermal efficiency. Since no undue force is exerted on the heater, there is no fear of breaking the heater.

The above-mentioned incense burner in accordance with another aspect of the present invention further comprises: a fixing plate, wherein an area of the heater is smaller than an area of the heating plate, the fixing plate stores and positions the heater so that the heater may not move laterally or rotate, and a peripheral part of the top cover positions the fixing plate so that it may not move laterally or rotate in the state of the fixing plate being stored in the opening of the top cover from its underside.

The heat of the heater should only be conducted to the container placed on the heating plate (in the upward direction). The area of the heater (size in the lateral direction) is selected to be smaller than the area of the heating plate. With this configuration, the heat of the heater is made hardly be conducted to the lateral direction, thereby to prevent the outer enclosure of the incense burner from becoming high temperature. It is also possible to prevent the user from the unintentional touch on the outer enclosure of the incense burner and possible burn.

In a case of selecting the area of the heater to be smaller than the area of the heating plate, a gap is created between the heater and the lateral part of the opening of the top cover. The fixing plate stores the heater (it may be the heater, and the first conduction plate and/or the second conduction plate), and position-regulating them so that the heater and the like may not move laterally or rotate. The fixing plate itself, in its state of being stored in the opening of the top plate from the underside) is position-regulated by the lateral part of the top cover so that it may not move laterally or rotate. With this configuration, an effect similar to the above-mentioned can be obtained, and at the same time, the heat of the heater is hardly conducted to the top cover. It is also possible to prevent the outer enclosure of the incense burner from becoming high temperature.

In the above-described incense burner in accordance with another aspect of the present invention, the heater cover has a bearing part on its peripheral part being in contact with a lateral part of the top cover, the bearing part is higher than a central part of the heater cover in which the heater is placed, and at least a part of a peripheral part of the heating plate is sandwiched between the bearing part and the top cover directly or through a rubber ring. With this configuration, the heating plate can be supported by the bearing part of the heater cover in a case where the heating plate is pushed from the outside. Any outside force is not directly conducted to the heater spring.

Preferably, the heating plate is sandwiched between the bearing part of the heater cover and the top cover, with a rubber packing inserted between the heating plate and the top cover. The rubber packing prevents the outside water or the aroma oil from intruding into the interior of the incense burner. Since the thermal conductivity of the rubber is low, the heat conducted to the heating plate is hardly conducted to the top cover.

Between the bearing part of the heater cover and the heating plate, there is provided a small gap which is selected to be not greater than the compression margin of the rubber packing. Even in a case where the heating plate is pushed downward from the outside, the heating plate stops by being supported by the bearing part of the heater cover. In such a case where the heating plate moves at its maximum, since the displacement of the heating plate is not greater than the compression margin of the rubber, the space between the top cover and the heating plate is maintained in a state where it is sealed by the rubber.

The above-described incense burner in accordance with another aspect of the present invention further comprises an outer lid-guard which has a plurality of openings and is provided for detachably covering the container, wherein a lower end of the outer lid-guard extends downwardly beyond a surface of the heating plate. With this configuration, it is possible to prevent the container supporting part from touching directly on the surface of the floor or the table when the container tumbles.

The incense burner in accordance with another aspect of the present invention comprises: an upper frame unit comprising on its top a heating body which heats an aroma material; a light source; a pillar part made from transparent or translucent material, having a pillar encircling the light source; an outer frame made from transparent or translucent material encircling an outer side of the pillar part; and a lower frame unit for covering a bottom side opening of the outer frame, wherein the outer frame is sandwiched between the upper frame unit and the lower frame unit, and the lower frame unit is fixed to the pillar part with a fastening member. With this configuration, it is made possible to enclose the part other than the part that serves to heat the material to be heated, and to prevent the user from his unintended touch on the heat source. In addition, by making the user notice the power supply to the heating body with an emission of the light source, and simultaneously performing the light art, it is possible to provide an incense burner that can comfortably be used with ease for long period of time.

In the above-described incense burner in accordance with another aspect of the present invention, the pillar part is attached to the upper frame unit by engagement prior to the fixing of the outer frame. By configuring it as described, it is possible to reduce number of members for fixing the pillar part, and at the same time, to reduce the occurrence of shadow on the inside wall of the outer frame, by reducing the unevenness of the inside of the incense burner when viewed from the outer frame.

In the above-described incense burner in accordance with another aspect of the present invention, the upper frame unit is configured with an upper frame and a heating body unit having the heating body, and the pillar part is provided with a plurality of arm parts with attachment pieces on its top, and is engaged with the heating body unit by making the arm parts with attachment pieces pass through a plurality of fitting holes provided in the heating body unit and then rotating the pillar part. With this configuration, it is possible to achieve a possible simplification of the assembled configuration of the incense burner.

In the above-described incense burner in accordance with another aspect of the present invention, the upper frame unit is configured with an upper frame and a heating body unit having the heating body, and the pillar part is fixed to the upper frame after being engaged with the heating body unit, thereby making its detachment from the upper frame unit unable. By doing so, it is possible to improve the assembling property of the incense burner.

In the above-described incense burner in accordance with another aspect of the present invention, each of the arm-attachment pieces provided on the pillar part is divided into two parts, one of the divided arm-attachment pieces is made to be easily bent at the time of fixing the lower frame unit to the pillar part with a fastening member. It takes a configuration, wherein the outer frame is sandwiched between the upper frame unit and the lower frame unit by fixing the lower frame unit to the pillar part (which is in engagement with the upper frame unit). The height of the upper frame unit, which is defined by being mounted on the top edge of the outer frame, is selected to be slightly higher than the height of upper frame unit, which is defined by the engagement with the pillar part. In the embodiment, by providing protrusions on parts of the upper frame unit which engage with the divided attachment pieces on the upper edge of the pillar part, a difference in the height is created (The higher the height of the protrusions is, the lower becomes the height of the upper frame unit defined by the engagement with the pillar part). By virtue of the difference in the height, the divided attachment pieces of the pillar part which engage with the upper frame unit bend upwards. The bending of the divided attachment pieces creates a reactive force in the direction of pulling the upper frame unit to the lower frame unit. By this reactive force, the upper frame unit and the lower frame unit catch the outer frame securely. In this manner, the outer frame is held securely.

In the above-described incense burner in accordance with another aspect of the present invention, the arm-attachment pieces on the pillar part are provided on a position outside of the position where the heating body unit is disposed. With this configuration, it is possible to make the pillar part being hardly influenced by the temperature rise of the heating body unit.

An incense burner in accordance with another aspect of the present invention, comprises: an upper frame unit having a heating body for heating an aroma material; a pillar part; an outer frame disposed outside of the pillar part; and a lower frame unit, wherein the pillar part is combined with the upper frame unit, and the pillar part is combined with the lower frame unit in a state where the outer frame is sandwiched between a peripheral part of the upper frame unit and a peripheral part of the lower frame unit. By doing so, it is possible to achieve a simplification of the assembled configuration of the incense burner.

In the above-described incense burner in accordance with another aspect of the present invention, the pillar part is engaged with the upper frame unit, by making a plurality of arm-attachment pieces provided on the pillar part pass through fitting holes provided on the upper frame unit and then rotating the pillar part, and at least a part of the plurality of the arm-attachment pieces bends in a state where the pillar part engages with the upper frame unit. By the reactive force created by bending, the upper frame unit and the lower frame unit catch the outer frame between them. By doing so, the outer frame is held securely.

The above-described incense burner in accordance with another aspect of the present invention further comprises: a control part, wherein the control part has a switch with a manipulation part which can control electric power supply to the heating body, and the pillar part is attached to the lower frame unit having a slider sliding part which includes a slider for operating the manipulation part of the switch, an under frame provided with a first urging member for urging the slider, and an under frame cover. With this configuration, it is possible to shut the power supply to the heating body off securely at the time when the main body falls down.

In the above-described incense burner in accordance with another aspect of the present invention, the slider contains, therein, a push rod, and a second urging member for urging the push rod to a direction of the manipulation part of the switch; a first urging member for urging the slider for operating the manipulation part of the switch is provided; and the second urging member serves to moderate an outside force exerted on the manipulation part of the switch, after the switch manipulation part reaches a limit of its movable range. By doing so, it is possible to prevent the switch main body from being exerted with an excessive load, even if the slider is operated beyond its normal movable range.

An incense burner in accordance with another aspect of the present invention, comprises: a heating body for heating an aroma material; a switch for controlling power supply to the heating body; a housing having an opening on its bottom; a slider partly stored in the housing for operating the switch directly or through a switch manipulation part and whose lower end projects downwards from the bottom opening; and a first urging member for urging the slider downwards, wherein in a state where the incense burner is placed on a plane, a lower end of the slider reaches a level of approximately the same as a bottom part of the housing and the switch permits power supply to the heating body and in a state where the incense burner falls down, the slider projects more from the bottom opening by being urged by the first urging member, compared with a referred state where the incense burner is placed on a plane, and the switch shuts off power supply to the heating body.

For instance, when the incense burner is brought to a state where nothing is existing under the incense burner, by holding the incense burner in the hand, the lower end of the slider project downwards from the lowermost face (typically, the lower faces of the legs of the housing) of the housing (in the embodiments, it includes the lower frame and the lower frame cover). In this state, the switch shuts off the power supply to the heating body. By installing the incense burner on a plane (typically, the floor, the shelf and the like), the plane pushes the slider upwards (in a direction of the slider for being stored in the incense burner). The lower end of the slider is brought to a level same as that of the lowermost face of the housing (the lowermost faces of the lower frame and the lower frame cover). In this state, the switch permits the power supply to the heating body.

The user uses the incense burner by installing it. The incense burner operates normally. If the user erroneously lets the incense burner to fall down laterally, the slider projects from the bottom of the housing by the first urging member, the power supply to the heating body is automatically shut off. According to the present invention, it is possible to shut off the power supply to the heating body securely at the time when the main body falls down.

In the embodiments, the incense burner takes a structure in which the switch manipulation part is pushed-in (C (Common) terminal and NO (Normally Open) terminal of the switch are in their conductive state) in case where the incense burner is installed on a plane, whereas the switch manipulation part is released (C (Common) terminal and NO (Normally Open) terminal of the switch are in their open state) in a case where the incense burner falls down. It may takes another structure in which the switch manipulation part is released (C (Common) terminal and NO (Normally Open) terminal of the switch are in their conductive state) in case where the incense burner is installed on a plane, whereas the switch manipulation part is pushed-in (C (Common) terminal and NO (Normally Open) terminal of the switch are in their open state) in a case where the incense burner falls down.

In the above-described incense burner in accordance with another aspect of the present invention, the slider comprises a slider main body; a push rod; a second urging member, one end of which is supported by the slider main body and the other end of which urges the push rod upwards, the housing comprises a slider stopper for limiting upward movement of the slider main body, the push rod and the second urging member are stored in the slider main body, and in a state where the slider main body is pushed upward to hit the slider stopper, the push rod is pushed upward by the second urging member, operates the switch to be in a conductive state, while maintaining resiliency of the second urging member.

In a structure in which the slider directly acts on the switch, there is an apprehension of deforming or breaking the switch or the like components, in a case of excessively pushing-in the switch. By providing a stopper on the slider, it is possible to prevent such an accident. It is however difficult to realize an optimum structure wherein the slider serves to its normal function while no undue load is exerted on the switch and the like components. In addition, there are some cases wherein the resiliency coefficient of the urging member that is optimum value for detecting the falling-down of the incense burner does not accord with the resiliency coefficient of the urging member that is optimum value for operating the switch.

In the present invention, the above-mentioned problem is solved by employing the first urging member and the second urging member. When the incense burner is placed on a plane, the plane pushes the slider upwards. The slider main body pushes the push rod upwards through the second urging member. The push rod acts on the switch directly or through the switch manipulation part. When the slider main body is pushed-in upward from the outside, the displacement of the slider is however limited by the slider stopper. The push rod makes the switch conductive, before the upper end of the slider main body hits the slider stopper, and the second urging member is in its movable range (maintains its resiliency), even in a state wherein the upper end of the slider main body hits the slider stopper. Since the push rod is stored in the slider main body, it is not possible to, at least easily, push the push rod directly from the outside. Preferably, the slider main body has a push rod stopper that limit the upward movement of the push rod. With this structure, it is possible to avoid an excessive load to be exerted on the switch main body, even if the slider is moved beyond the normal operating range. The resiliency coefficient of the first urging member is set to its optimum value for detecting the falling-down of the incense burner. And, the resiliency coefficient of the second urging member is set to its optimum value for operating the switch.

In the above-described incense burner in accordance with another aspect of the present invention, the control part is held by the pillar part and the lower frame unit. With this configurations, it is possible to hold the control part securely without increasing the number of components.

### DESCRIPTION OF THE PREFERRED EMBODIMENT

In the following paragraphs, description will be made on the embodiments showing the best mode for concretely embodying the present invention with reference to the drawings.

<<Embodiment 1>>

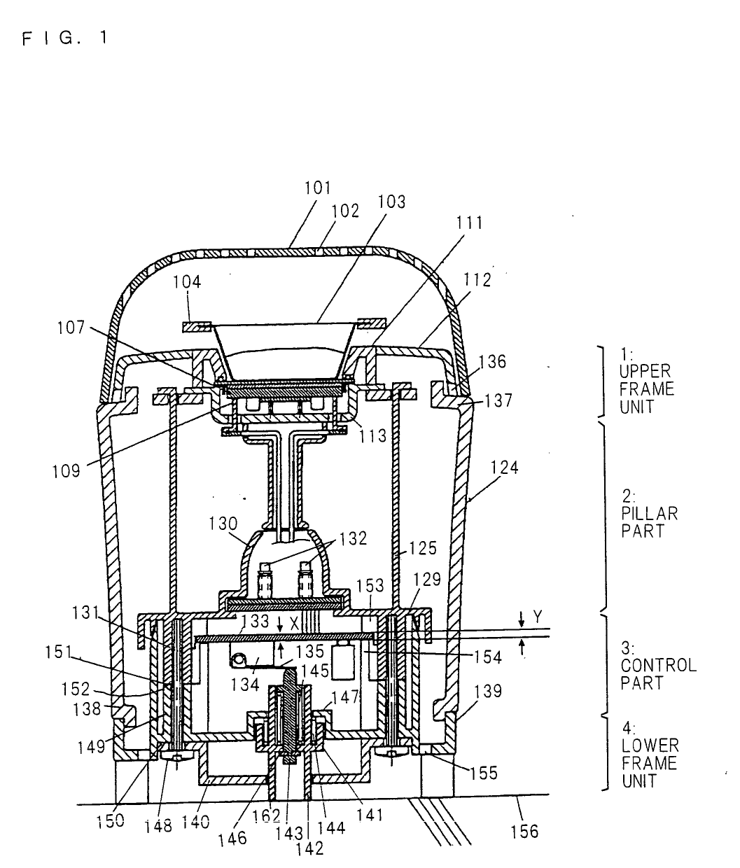

In the followings, the incense burner of Embodiment 1 of the present invention will be described with reference to FIG. 1 through FIG. 10. FIG. 1 is a cross-sectional view of the essential configuration of the incense burner in Embodiment 1.

The incense burner is configured with an outer lid-guard 101, a container 103, an upper frame unit 1, a pillar part 2, a control part 3 and a lower frame unit 4. An exterior enclosure of the main body is composed of the outer lid-guard 101 for covering the upper part of the main body, an outer frame (body) 124 for forming the side face of the main body and a lower frame 139. The incense burner according to the present invention is placed on installation plane 156.

The outer lid-guard 101 is provided for preventing a user of the incense burner from an easy access or touch to the container 103, when green tea leaves are placed in the container 103 and the heater (heating body) 107 is caused to generate heat. The outer lid-guard 101 is designed to be detachable from the main body and has a plurality of openings 102, and made from polyethylene terephthalate (PET) resin and/or polybutylene terephthalate (PBT) resin. The lower end of the outer lid-guard 101 extends downwards to a level under the level of a surface of the heating plate 105.

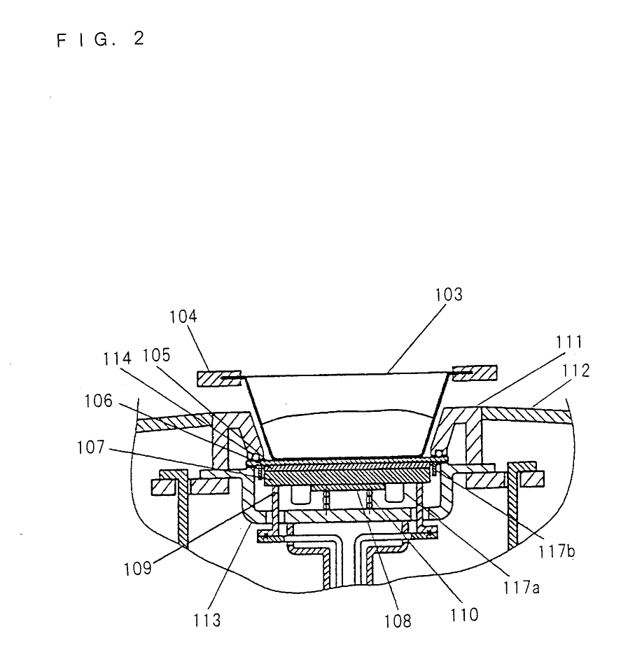

FIG. 2 is an enlarged view of a part around the heater of Embodiment 1 of the present invention. Numeral 103 designates a container for accommodating the material to be heated, in this Embodiment, 2 g of green tea leaves, and for being mounted on a heating plate 105. The container 103 has a square containing part made of stainless steel and a handle part 104 made from a resin for surrounding the containing part. The handle part 104 is joined to the stainless steel containing part only at its four corners for insulating heat conduction.

Numeral 107 designates a PTC (Positive Temperature Efficient) heater having a self-temperature regulating function, which is a device having a Curie temperature of 260° C. On the heater 107, there is mounted a first conduction plate (conduction plate A) 106 made of aluminum. On the first conduction plate 106, there is mounted a heating plate 105 made of stainless steel. On both the boundaries between the heater 107 and the first conduction plate 106, and between the first conduction plate 106 and the heating plate 105, there is painted a heat dispersing compound (the compound is not shown in the drawing) for improving the heat conduction.

Electric poles 109 are attached to the heater 107. When a power switch provided on the main body of the incense burner turns ON, a power is supplied to the heater 107.

Numeral 108 designates a second conduction plate (conduction plate B) provided between a heater spring 110 and the heater 107 for maintaining a thermal uniformity of the under face of the heater 107. The heater spring 110 is a coil spring for urging the heater 107 to the direction of the heating plate 105.

Numeral 111 designates a top cover provided for covering the heating plate 105. The top cover 111 has a vertically piercing opening, which has an inner fringe part provided along the periphery of the opening for holding the heating plate 105 so that it may not escape upwards, and a lateral part for holding the heating plate 105 and the heater 107 stored in the opening from the bottom and for regulating (positioning) positions of these components directly or through a fixing plate so that they may not move laterally or rotate. The top cover 111 is joined to an upper frame 112 and forms an upper part of the main body together with the upper frame 112.

Numeral 113 designates a heater cover on which one end of the heater spring 110 rests. The heater cover 113 is held by the top cover 111 by rotating it. In the center of the heater cover, there is provided a concave part (not shown) for the heater spring 110 to be inserted. The top cover 111 and heater cover 113 are made from polyphenylene sulfide (PPS) resin having a high heat-resistant property, because they cover the heater 107. In order to prevent a thermal discoloring of the PPS resin, it is desirable to select a dark color for the resin, and black is used in this Embodiment 1.

In an approximately closed space defined by the top cover 111 and the heater cover 113, the heater spring 110, the second conduction plate 108, the heater 107, the first conduction plate 106 and the heating plate 105 are stored.

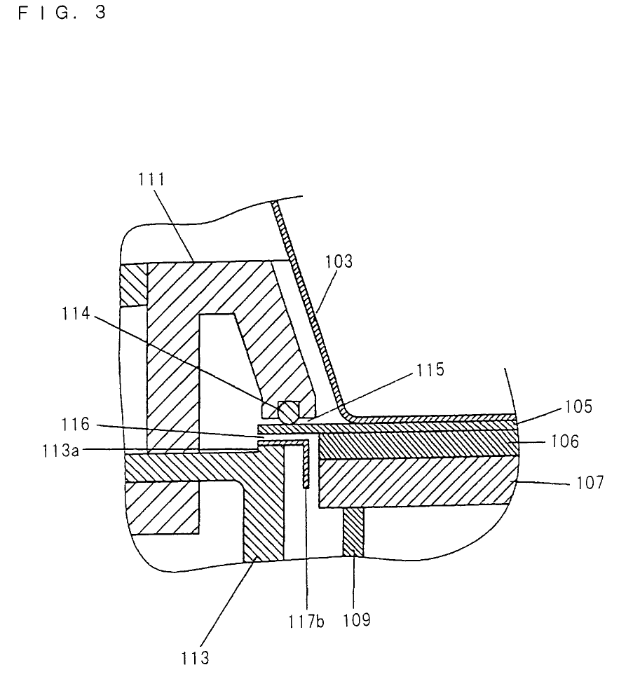

FIG. 3 is a cross-sectional view around a rubber ring of Embodiment 1 of the present invention. Numeral 114 designates the rubber ring made from a silicone rubber (rubber for packing), being inset in and along a groove provided on the top cover 111, and sandwiched between it and the heating plate 105, for preventing any liquid dropped on the heating plate from intruding into the interior of the incense burner. The silicone rubber ring 114 is held between the top cover 111 and the heating plate 105, while providing a space 115 between the heating plate-side tip end of the top cover 111 and the heating plate 106.

The heater cover 113 is provided with a bearing part 113a for supporting the heating plate 105, thereby providing a gap 116 between the bearing part 113a of the heater cover 113 and the heating plate 105, and the gap 116 is set to be not greater than the compression margin of the silicon rubber ring 114. The heating plate 105 is set to position above the upper opening of the heater cover 113 through the gap 116.

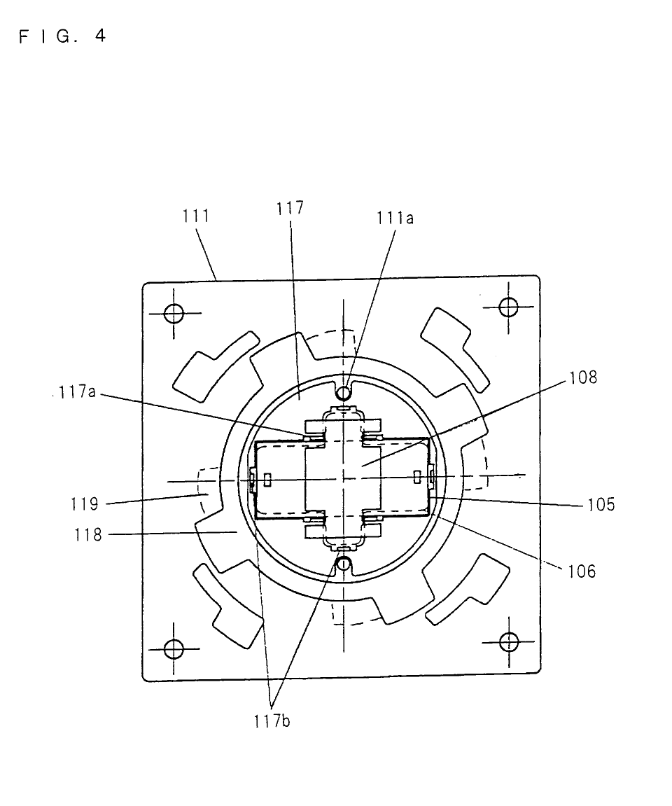

FIG. 4 is a bottom view of the heating body unit of Embodiment 1 of the present invention while it is being assembled. Numeral 117 designates a fixing plate positioned by protrusions 111a provided on the top cover 111. The heater 107 and the first conduction plate 106 are vertically fixed by the heating plate 105 and the heater spring 110, and their horizontal positions are determined by the fixing plate 117. The fixing plate 117 is a disk made of stainless steel having a central opening for storing the heater 107 and the first conduction plate 106. The fixing plate 117 has, along the periphery of its opening, four downwardly extending thin-plate ribs 117a and four small plate ribs 117b. It positions the first conduction plate 106 and the heater by the downwardly extending plate ribs 117a and 117b, and positions the heater 107 by the further downwardly extending parts of the plate ribs 117a and 117b. The second conduction plate 108 is positioned by the plate ribs 117a of the fixing plate 117.

The fixing plate accommodates the heater and positions the heater so that it may not move laterally or rotate, while the peripheral part of the opening of the top cover, in its state of storing the fixing plate, positions the fixing plate so that it may not move laterally or rotate.

The heater cover 113 is provided with four legs (not shown), and the top cover 111 is provided with four grooves 118 into which the four legs of the heater cover 113 are inserted, and four fit-parts 119 for holding them after the legs are inserted and rotated. The heater cover 113 is engaged with the top cover 111 by being rotated.

In the following description, a structure created by enclosing the heating plate 105 and the heater 107 with the top cover 111 and the heater cover 113 is referred to “heating body unit 11”. A structure created by adding the upper frame 112 to the heating body unit 11 is referred to as “upper frame unit 1”.

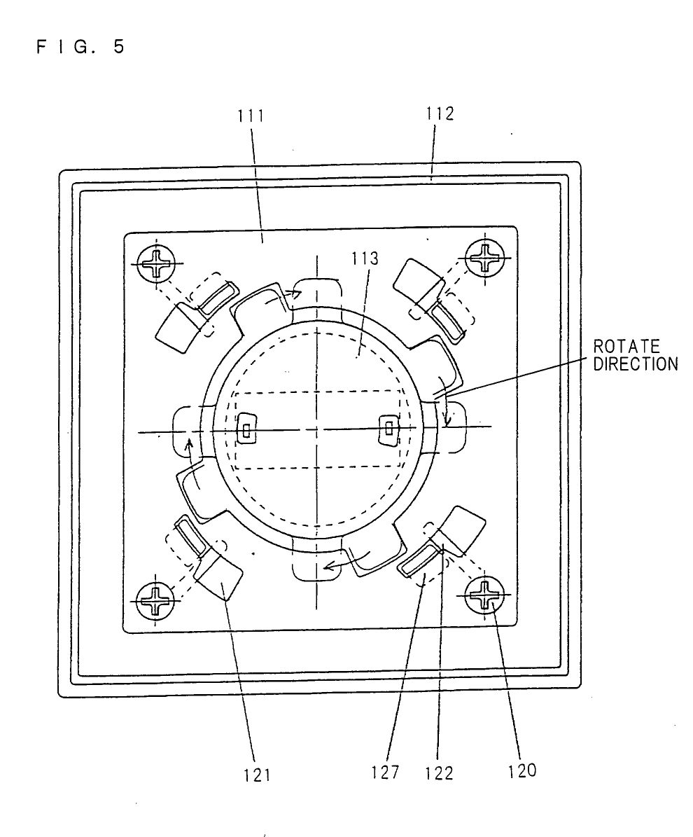

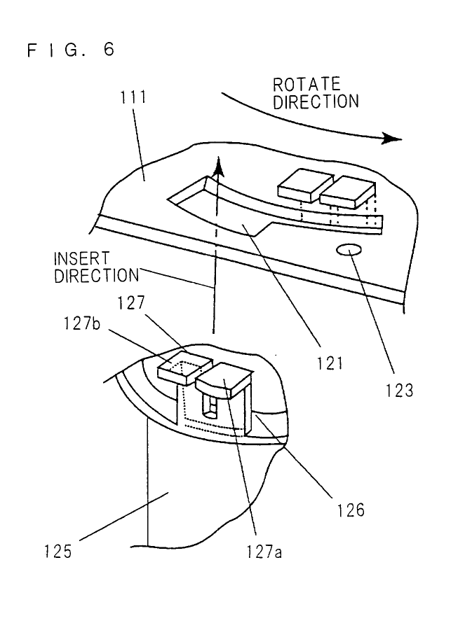

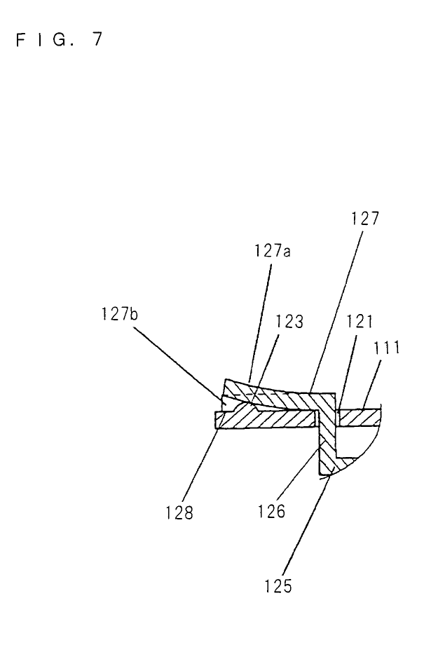

FIG. 5 is a bottom view of the upper frame unit in Embodiment 1 of the present invention. FIG. 6 is an oblique view of the essential components for showing a state of attachment of a pillar 125 to the upper frame unit. FIG. 7 is a cross-sectional view of the essential components for showing the state of attachment of a pillar 125 to the upper frame unit.

The top cover 111 has, on its four corners, fitting-hole parts 121. The pillar 125 is engaged with the top cover 111, by causing attachment pieces 127 of arm parts provided on the top of the pillar 125 to pass through the fitting-hole parts 121 and then to rotate relatively. Each of the attachment pieces 127 of the pillar 125 is divided into two parts (127a and 127b). The top cover 111 includes protrusions 123 at the positions upon which the attachment pieces 127a rest. When a load is applied on the pillar 125 by pulling it downwards, the attachment pieces 127b are engaged with the engagement parts 128 provided on the top cover 111, thereby causing the attachment pieces 127 to be fixed in their states where reactive forces are generated there in compliance with the heights of the protrusions 123.

By fixing the lower frame unit 4 to the pillar part 2 (which is engaged with the upper frame unit 1), a structure of sandwiching the outer frame 124 between the upper frame unit 1 and the lower frame unit 4 is created. The height of the upper frame unit 1, defined by being mounted on the top fringe of the outer frame 124, is set to be slightly higher than the height of the upper unit 1 defined by its engagement with the pillar part 2. In the Embodiment, the difference in the height is created by providing the protrusions 123 on the parts of the upper frame unit 1, which are in engagement with the divided attachment pieces 127a of the pillar part 2 (The higher the height of the protrusions 123 is, the lower becomes the height of the upper frame unit 1 which is defined by the engagement with the pillar part 2). By being caused by this difference in the height, the divided attachment pieces 127a of the pillar part 2 which are in engagement with the upper frame unit 1, bend upwards.

The bending of the attachment pieces 127a of the pillar 125 which are in engagement with the top cover 111 by the protrusions 123, increases the power in the direction for sandwiching the outer frame 124 between the upper frame unit 1 and the lower frame unit 4, thereby making the holding of the outer frame 124 more secure.

The upper frame 112 has rotation stoppers 122. In this Embodiment, the top cover 111 is fixed to the upper frame 112 with screws 120 after the pillar 125 is engaged with the top cover 111. By prohibiting the rotation of the pillar 125 by the stoppers 122 of the upper frame 112, a structure is created where the detachment of the pillar 125 from the upper frame unit 1 is not possible.

In FIG. 1, numeral 124 designates an outer frame for surrounding the outside of the pillar part 2 made from a transparent or translucent material, and its inside face is finished to have a fine ruggedness. The top fringe part 137 of the outer frame 124 forms upper openings 136 by being partly in contact with the upper frame unit 1. Under the bottom fringe part 138, there is provided a lower frame 139 for covering the bottom open end of the outer frame 124.

The pillar part 2 is configured with the pillar 125 made from a transparent or translucent material for covering the light source 132, an enclosure part 129 for storing a base board 133, a light source cover 130 for covering the light source 132, and bosses 131 on which fastening members 148 such as screws are attached. The numeral 132 designates the light source such as LED whose turns ON/OFF states are regulated by the control part 3.

The control part 3 includes the base board 133, a switch 134 for regulating the power supply to the heating body and a switch manipulation tip 135.

The lower frame unit 4 is configured with the lower frame 139 and a lower frame cover 140. The lower frame 139 has openings 155 on its bottom. Inside the lower frame unit 4, there is provided a slider sliding part 141 for enabling a slider 142 to slide therein. The slider 142 operates the switch manipulation tip 135 of the switch 134 mounted on the base board 133. The lower end of the slider 142 project downwards from the bottom opening of the lower frame unit 4. The slider 142 is urged to a side for shutting the power supply to the heater 107 by a first urging member 144 for releasing the switch manipulation tip 135 and thereby bringing the switch 134 to a state of “OPEN”.

The slider 142 includes a slider main body 162, a push rod 143, and a second urging member 145, one end of which is supported by the slider main body and the other end of which urges the push rod upwards. Inside the slider main body 162, the second urging member 145 which urges the push rod 143 to the direction of the switch manipulation tip 135 (upward direction) is stored. Numeral 146 designates an E-ring for fastening the push rod 143 to the slider 142. Numeral 147 designates a slider stopper provided inside the slider sliding part 141. The slider stopper 147 provided in the lower frame 139 (a part of the housing) limits the upward movement of the slider main body 162.

In a state wherein the slider main body 162 is pushed upwards to hit on the slider stopper 147, the push rod 143 is also pushed upwards by the second urging member 145 for operating the switch 134 to its conductive state, while maintaining the resiliency of the second urging member 145. In this Embodiment, the first urging member 144 and the second urging member 145 are springs.

The fastening members 148 pierce through the through holes A 149 of the lower frame 139 and the through holes B 150 of the lower frame cover 140, and are fixed to the bosses 131 of the pillar part 2. A gap has been provided between the bottom ends 151 of the bosses 131 and the ends 152 of the through holes A 149 of the lower frame 139 before the attachment of the fastening members 148, but the both are closely in contact with each other after the attachment.

The base board 133 is held by being sandwiched between the control part holding ribs 153 provided in the storing part 129 of the pillar part 2 and the control part holding bosses 154 being close to the through holes A 149 of the lower frame 139. Since the gap formed by the ribs 153 and the bosses 154 is set to larger than the thickness X of the base board 133, it will not inhibit the operation, at the time of assembling, of bringing the top faces 151 of the bosses 131 of the pillar part 2 to contact closely with the end faces 152 of the through holes A 149 of the lower frame 139. The control part 3 is so configured that it can be held stably simultaneously when the lower frame unit 4 is attached to the pillar part 2.

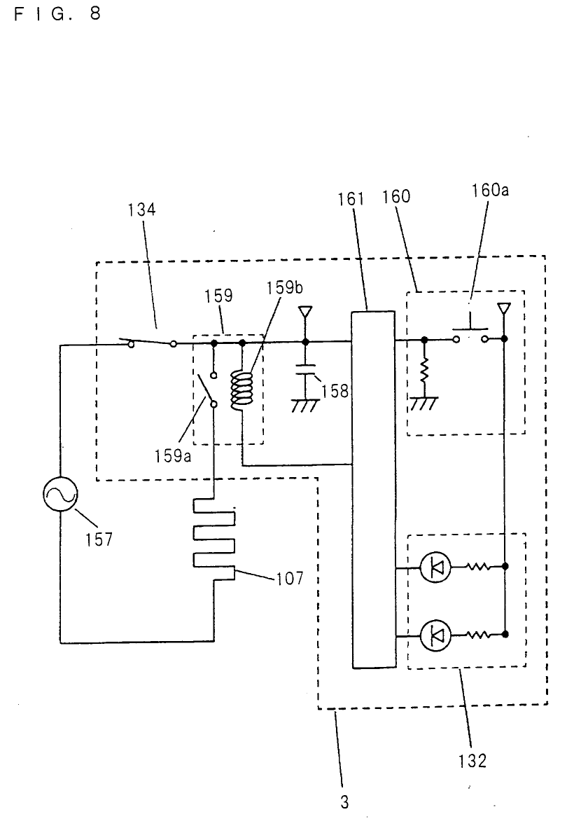

FIG. 8 shows a circuit diagram of the control part 3 of Embodiment 1. In FIG. 8, numeral 157 designates an AC power source and numeral 158 designates a DC power source. The heater 107 is activated with the configuration of the AC power source 157 and a relay 159 with a relay contact point 159a serially connected to the AC power source 157 and a relay coil 159b for controlling the relay contact point 159a, wherein the contact point 159a is closed by making a current flow through the relay coil 159b.

A current-conducting switch 160 can perform opening and closing of the relay contact point 159a by pushing a switch 160a for actuating the relay 159.

Numeral 132 designates a light source which adopts a plurality of LEDs. Numeral 161 designates a microprocessor which executes a program for causing the LEDs 132 to emit light at a particular period. A switch 134 makes its contact point normally “CLOSE” by the push rod 143 provided on the slider 142.

The incense burner of Embodiment 1, thus configured, will now be described on its operation. The aroma material, the green tea leaves, is put on the container 103 which is then mounted on the heating plate 105. After setting the outer lid-guard 101 thereon, the power switch 160 is turned ON. The ON state of the power switch causes the heater 107 to be heated. The heat of the heater 107 is conducted to the heating plate 105 and the containing part of the container 103, the both are made of a metal, with a good efficiency, through the first conducting plate 106 of good heat conductivity, thereby to cause the green tea to generate its aroma component.

The incense burner thus prepared is heated up to 260° C., which is the Curie Point, by employing the PTC heater 107 having a self-temperature regulating function. And the heating plate 105 is also heated up to a temperature sufficient for generating the aroma component from the green tea or black tea leaves. The temperature of the outer enclosure of the main body can however be suppressed, because the heat is concentrically conducted from the heater 107 to the heating plate 105 through the first conducting plate 106.

By covering the heater 107 with the top cover 111 and the heater cover 113 as shown in this Embodiment, the temperature rise at the outer enclosure of the main body is further suppressed. Although the PTC heater is employed for the heater 107 in Embodiment 1, any kind of heater (suitably selected heater) can heat the green tea or black tea leaves up to a temperature for causing them to emit the aroma component, as long as the configuration of Embodiment 1 is adopted, and, it is possible to efficiently suppress the temperature rise at the outer enclosure of the main body. In Embodiment 1, although a stainless steel is used for forming the heating plate 105 and the container 103, any metal with a high heat-conductivity such as aluminum may be employed for the purpose.

Although aluminum has a good heat-conductivity, it however has a too large radiation, and thus, if it is employed for the heating plate 105 and the container 105, there are some instances of lowering the temperature.

In Embodiment 1, by employing stainless steel for the heating plate 105 and the container 103, it is possible to more efficiently conduct the heat to the tea leaves.

Further, since the first conducting plate 106 made of aluminum with high heat conductivity is used for being sandwiched between the heater 107 and the heating plate 105, the heat generated from the heater 107 can concentrically be conducted to the heating plate 105. In contrast, to the horizontal direction from the heater 107, the heat is however conducted only through the limited contacts of the plate ribs 117a and 117b of the fixing plate 117. As a result of this measure, the temperature rise of the fixing plate 117 is suppressed, and thus, temperature rises of a part of the periphery of the heating plate 105 where the fixing plate 117 contacts, of the silicon rubber ring 114 which contacts the heating plate 105 at its periphery, and of the top cover 111 which positions the fixing plate 117, are also suppressed.

Any shape and material can also be employed for the fixing plate 117 of Embodiment 1, as far as they can insulate the heat conduction, and thus, are not limited to those used in this Embodiment. However, the fixing plate 117 requires a heat-resistant property and takes a complex shape for fixing a plurality of components, and thus, is desirably made of stainless steel as in Embodiment 1.

In this Embodiment, the fixing plate 117 is positioned by the top cover 111. Instead, it is also possible to position the fixing plate by the heater cover 113, by providing similar ribs on the heater cover 113. In this case however, it is not possible to cause the heater cover 113 to engage with the top cover 111 by rotating the heater cover 113. The heater cover 113 is attached to the top cover 111 by the use of screws and the likes.

By attaching the silicon rubber ring 114 to the top cover 111 between the heating plate-side tip of the top cover 111 and the heating plate 105 in a state where there is provided a gap between them, the components can be sealed at a position where the temperature of the top cover 111 is lowered as much as that is provided by the gap 115 on the heating plate 105. Further, although the heat of the heating plate 107 is conducted through the heater spring 110 to the heater cover 113, the heat conduction to the heater cover 113 is however suppressed, because a coil spring is used as the heater spring 110.

In addition, any other type of springs, such as a leaf spring and the like, may also be employed for obtaining the same effect, if it only has the object of urging the heater 107. Further, even if the heating plate 105 is pushed with a much more urging force than that derived from the heater spring 110, it is possible to prevent an exertion of an excessive force on the heater 107, because the heater cover 113 is provided with a bearing part 113a, on which the force is borne.

Moreover, by providing a gap 116 between the bearing part 113a of the heater cover 113 and the heating plate 105, and setting the gap 116 to not greater than the compression margin of the silicone rubber ring 114, it is possible to maintain the sealing between the heating plate 105 and the top cover 111 to a range of elongation of the silicone rubber ring 114, even if the heating plate 105 is lowered by being pushed with a force.

In Embodiment 1, although the second conduction plate 108 is employed in order to secure the thermal uniformity of the heater 107 and to prevent a possible cracking at cooling, it is possible to prevent the temperature rise of the fixing plate 117 and to improve the assembling property, by positioning the second conduction plate 108 by the plate ribs 117a of the fixing plate 117 in the same manner as those for the heater 107 and the first conduction plate 106, and simultaneously with the latter. In addition, although the heating plate 105 is employed for being in service for a more long time with safety in Embodiment 1, the performance of the heater 107 can be secured without the heating plate 105.

In Embodiment 1, the assembling property of the incense burner is improved by inserting the heater spring 110 in the central convex rib of the heater cover 113 and fixing it therein.

Further, the assembling property is also improved due to the fact that the heater cover 113 is held by the top cover 111 by inserting the former into the latter and then rotating them, thereby causing the heater spring 110 to be fit therein without escaping therefrom, while the heater spring 110 is effectively urging the heater 107 and the heating plate 105.

In addition, although the top cover 111 is joined to the heater cover 113 by engagement without using a screw in Embodiment 1, it is also possible to fasten them with the screws after the insertion, or to assemble them only by fastening them without performing any insertion and rotation.

Further, by providing the outer lid-guard 101, it is possible to prevent unintentional touch of the user on at least the heating plate 105 and the container 103, and to prevent the user from possible burn. In addition, by configuring the outer lid-guard 101 to be detachable and to extend downwards below the surface of the heating plate 105, the container 103 is made to be stored in the outer lid-guard 101 at the time of falling down of the incense burner main body, and thus it is possible to prevent the container from rolling out of the main body to directly touch on the surface of the floor or the table.

In addition, although the outer lid-guard 101 is designed to cover the upper frame 112 in Embodiment 1, it is not necessary to cover the entire upper frame 112, as long as the lower end of the guard sufficiently extends downwards below the surface of the heating plate 105. And the effect of Embodiment 1 is obtained without the outer lid-guard 101, except for the possible hazard of burning and the unintentional touch of the container 103 at the time of falling down.

When the power switch 160 turns ON, the heater 107 starts to generate heat, and at the same time, the light source 132 starts to emit the light. The state of the light emission of the light source 132 can be observed from the outside through the light source cover 130, the pillar 125 and the outer frame 121, each made from the transparent material.

When the heater 107 generates heat for heating the container 103, the heating body unit 11 generates heat. Since the heating body unit 11 is covered by the outer frame 123 at its lateral side and by the upper frame 112 at its top, it is so configured in that it will not permit the easy access and touch on the heating body unit 111 by the user during the time of power supply.

Since the pillar 125 is engaging with the top cover 111 at a position apart from the heater 107, the pillar is designed to be hardly influenced by the heat of the heater 107. The arm parts 126 of the pillar 125 are therefore hardly enduring the possible deterioration due to the aging by heat. Since the pillar 125 is attached to the top cover 111 by rotating it, it is possible to make its shape simple inside the outer frame 124 (in the shown Embodiment, a cylindrical shape with no unevenness). As a result, it is possible to reduce the possible projection of the shadow of the pillar 125 on the outer frame 124.

During the power supply, the inside space of the outer frame 124 is heated by the heating body unit 11. Since the air taken-in from the openings 155 of the lower frame 139 is exhausted from the openings 102 of the outer lid-guard 101 through the gaps 136, it is possible to reduce the rise in the temperature of the outer frame 124 brought by the heat conduction from the heating body unit 11.

The heat generation by the heater 107 continues until the manipulation of the power switch 160, or until the lapse of a predetermined time set at the microprocessor. During this time, the user can therefore enjoy the aroma of the aroma material and the light of the light source 132 with ease.

In a state where the incense burner is placed on a plane, the lower end of the slider 142 is at a level approximately the same as the bottom face of the lower frame unit 4 (the bottom faces of the legs of the lower frame 139). In this state, the switch 134 permits the power supply to the heater (heating body) 107.

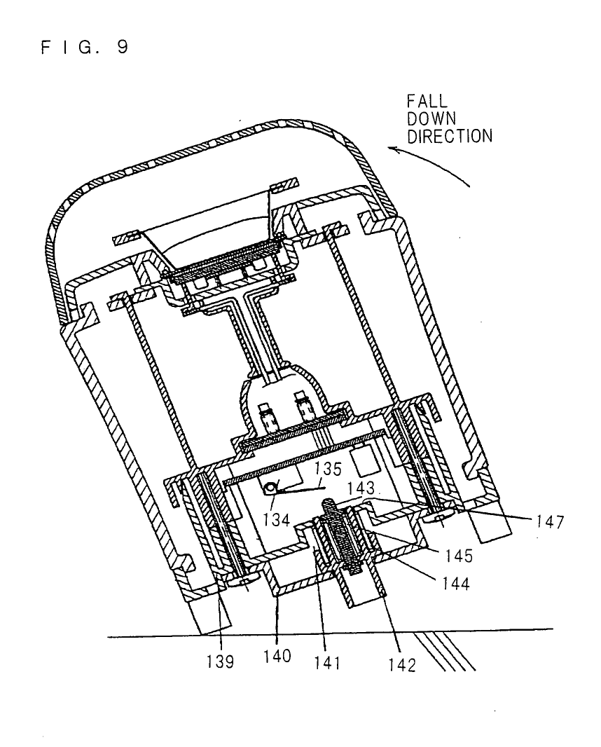

FIG. 9 is a view showing the state at which the incense burner of the present invention falls down. In a case where the incense burner unexpectedly falls down during its use, the slider 142 urged by the first urging member 144 projects from the bottom face of the lower frame unit 4 and releases the switch manipulation tip 135, thereby to bring the switch 134 to its state “OPEN”. The switch 134 shuts the power supply through the heater (heating body) 107 off. The slider 142 and the first urging member 144 constitute a safety device for stopping the power supply through the heater 107. The movable range of the lever 142 is regulated by the slider sliding part 141 configured with the lower frame 139 and the lower frame cover 140.

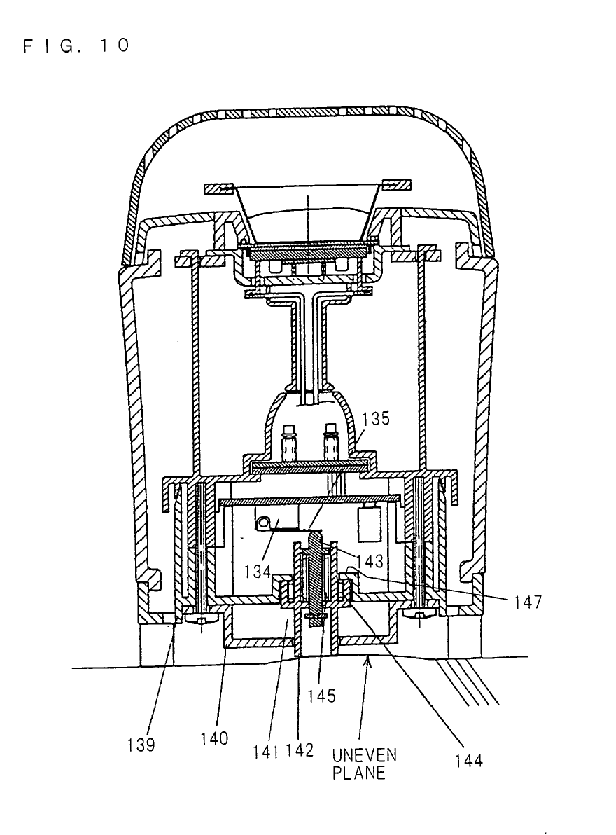

FIG. 10 is a view showing the state at which the incense burner of the present invention is placed on an uneven installation plane. It indicates the state where the slider 142 is limited its pushed-in movement by the slider stopper 147 when the incense burner of the present invention is placed on an uneven installation plane. The slider stopper 147 serves not to exert a too much load on the switch 134 and the base board 133 in the control part 3 by the push rod 143 inside the slider 142, by the intended function of the second urging member 145. By this provision, the normal use is made possible.

<<Embodiment 2>>

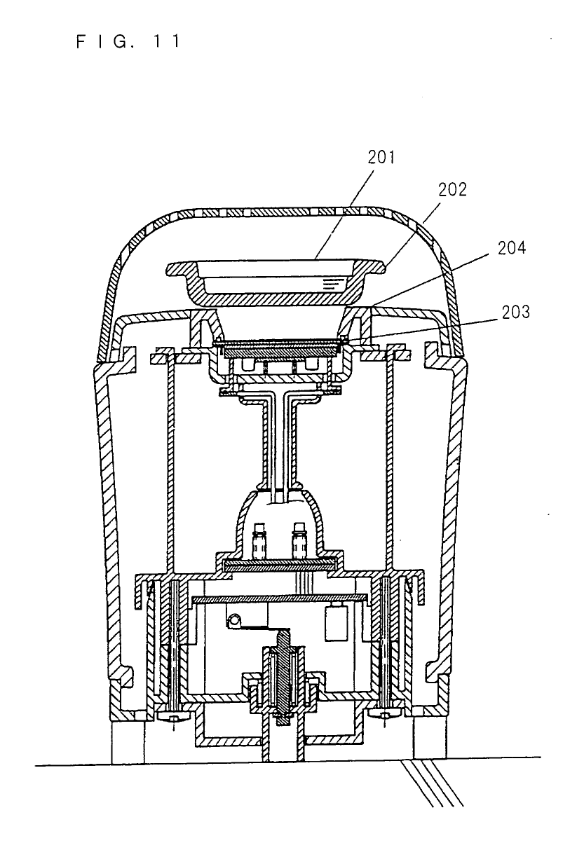

The incense burner of Embodiment 2 of the present invention will be described with reference to FIG. 11. FIG. 11 is a cross-sectional view of the essential configuration of the incense burner of Embodiment 2. The incense burner of Embodiment 2 is different from the incense burner in Embodiment 1 (having the container 103) only in its container 201. Except for this, they are the same.

The container 201 is made of a china wear and has a handle part 202 at is peripheral part, and usually accommodates 8 g of water and 2 drops of essential oil which are the material to be heated. Numeral 203 designates a heating plate. Numeral 204 is a top cover covering the heating plate 203 and is made from PPS resin in order to secure a sufficient heat-resistant property against the heat from the heating plate 203 and the container 201. The container 201 is mounted on the top cover 204 which covers the periphery of the heating plate 203. The following configuration and operation are approximately similar to those of Embodiment 1, but different therefrom in the points of the material for the container 201, and of its manner of warming the container by the use of atmosphere above the heating plate 203 and by the heat conduction from the top cover 204. In the thus prepared incense burner, the temperature of the container 201 is extremely lowered relative to that of the heating plate, because the material for the container 201 has a low heat-conductivity, and the heat from the heater and the heating plate 203 is conducted through the air, the resin and the rubber, each having a low heat-conductivity. By configuring the incense burner as described in the above, it is possible to obtain an optimum temperature in the container 201 for the purpose of enjoying the aroma from the aroma oil.

In Embodiment 2, the container 203 is mounted on the top cover 204. It may also be mounted on any place other than the top cover 204, as long as it can be mounted while holding a space between the container 201 and the heating plate 203. However, it is the most preferred to mount the container 201 on the top cover 204 made from a heat-resistant resin.

By combining the container 103 and the other container 201, each having a different shape, with the main body of the incense burner of this Embodiment (the incense burner main bodies of Embodiment 1 and Embodiment 2 are the same), it is possible to provide an incense burner which can comfortably be used in a plurality of temperature ranges.

Although the china wear is selected for the material of the container 201 in this Embodiment 2, any other material may be employed as far as it has a low heat-conductivity, and thus porcelain or steatite may also be used.

<<Embodiment 3>>

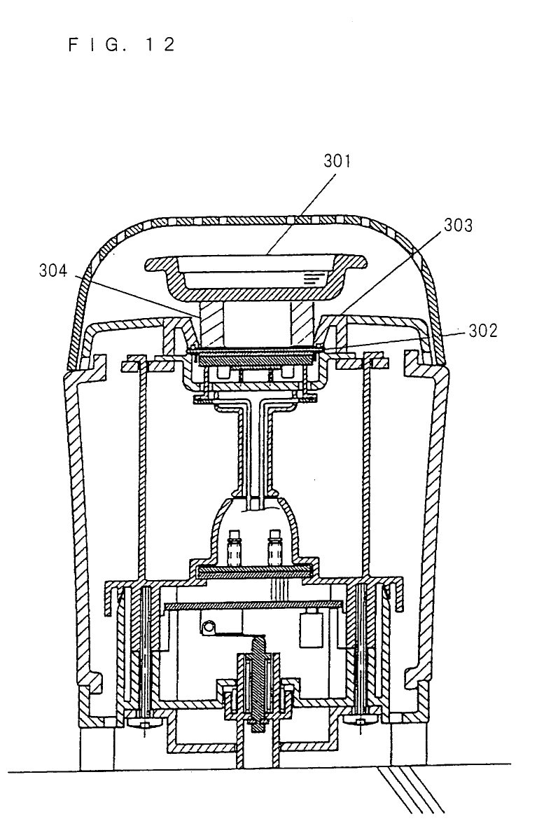

In the following paragraph, the incense burner in compliance with Embodiment 3 of the present invention will be described by referring to FIG. 12. FIG. 12 is a cross-sectional view of the essential configuration of the incense burner of Embodiment 3. Numeral 301 designates a container accommodating 8 g of water and 2 drops of essential oil which are the material to be heated. Numeral 302 designates a heating plate and numeral 303 designates a top cover. Numeral 304 designates legs for providing a space between the bottom of the container 301 and the heating plate 302.

The configuration and the operation of the incense burner in Embodiment 3 are different from those in Embodiment 2 in the point of provision of the legs 304 on the container 301, whereby the legs 304 are mounted on the heating plate 302. In other points, Embodiment 3 is the same as Embodiment 2. In the thus prepared incense burner, the heat from the heating plate 302 is conducted to the container 301 through the legs 304. By designing the cross-sectional areas and lengths of the legs 304 at their optimum, it is possible to select the temperature inside the container 301 and the temperature of the atmosphere above the heating plate 302 to their optimum values. Air flows among the legs 304 of the container 301. Since the air performs convection without being kept under the container 301, this air robs the heating plate 302 of the unnecessary heat of the heating plate 302 and does not unduly heat the top cover 303. Thus, it is possible to prevent the conduction heat from the top cover 303. Further, by configuring the legs 304 to those shown in FIG. 12, so that the peripheral ends of the legs 304 may rest on the inner periphery of the top cover 303, it is possible to hold the container 301 by the top cover 303 at the time of inclining or moving the incense burner main body.

<<Embodiment 4>>

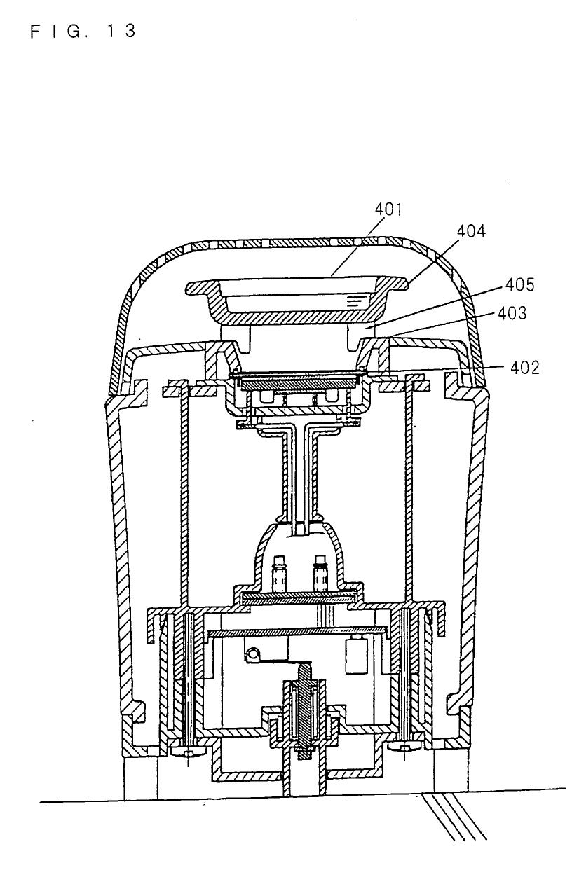

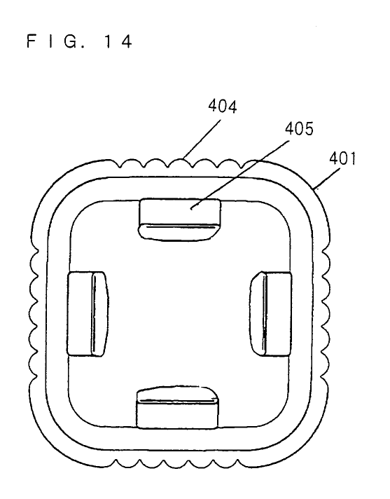

In the following paragraph, the incense burner in compliance with Embodiment 4 of the present invention will be described by referring to FIGS. 13 and 14. The incense burner in Embodiment 4 is different from that of Embodiment 2 (having the container 301) only in the provision of the container 401, and is the same in other points. FIG. 13 is a cross-sectional view of the essential configuration of the incense burner of Embodiment 4. FIG. 14 is a bottom view of the container of Embodiment 4. Numeral 401 is a container accommodating 8 g of water and 2 drops of essential oil which are the material to be heated. Numeral 402 designates a heating plate and numeral 403 designates a top cover. The container 401 has handle parts 404 on its peripheral part and legs 405 on its bottom for providing a space between the top cover 403 and the bottom of the container 401. The container 401 is retained on the top cover 403 with the legs 405.

In the thus prepared incense burner, it is possible to make the temperature inside the container 401 and the temperature of the atmosphere above the heating plate 402 optimum, by suitably designing the cross-sectional areas and the lengths of the legs to their optimum values. Further, since the container 401 is retained on the top cover 403 at its inside edge, it is possible to prevent any scratch on the heating plate 402.

By configuring the incense burner so that the outer peripheries of the legs 405 can engage with the inner periphery of the top cover 403, the container 401 is retained on the top cover 403 in the case where the incense burner main body inclines or moves. Further, as shown in FIG. 14, by providing the legs 405 in the direction of the handle parts 404, it is possible to prevent the temperature rise of the handle parts 404, because the atmosphere above the heating plate 402 is effectively shut off by the legs 405.

As described in the above, in accordance with the present invention, it is possible to obtain an advantageous effect of realizing an incense burner that effectively prevent the user from easy touch on its high temperature part.

In accordance with the present invention, it is possible to obtain an advantageous effect of realizing an incense burner that can heat material such as green tea leaves or black tea leaves to a temperature sufficient for generating their aroma components while suppressing the temperature rise of the outer enclosure.

In accordance with the present invention, it is possible to obtain an advantageous effect of realizing an incense burner that can comfortably be used at a plurality of temperature ranges.

In accordance with the present invention, it is possible to obtain an advantageous effect of realizing an incense burner that can comfortably be used for a long period of time with ease, by configuring it so that it may notice the power supply state of the heater (heating body) to the user by the emission of the light source, and at the same time, can perform a light art.

While the present invention has been described with reference to the preferred embodiments to a certain degree of its detail, the disclosed contents of the preferred embodiment may be varied at the particular detail of the configuration, and thus, the combination of the respective parts and components, and the change in the order of combination can be realized without departing from the scope and spirit of the invention.

1. An incense burner comprising: a container for accommodating material to be heated; a heating plate disposed beneath said container; a heater for heating said heating plate; and a supporting member for supporting said heating plate.

2. The incense burner in accordance with claim 1, wherein said container includes a set of a first container and a second container which have different shapes and are interchangeably mountable on said heating plate; said first container is so disposed that its bottom being in substantial contact with said heating plate; and said second container is so disposed that its bottom being spaced apart from said heating plate.

3. The incense burner in accordance with claim 2, wherein said second container is mounted on a top cover which covers a periphery of said heating plate.

4. The incense burner in accordance with claim 2, wherein said second container has at least one leg for providing a space between said heating plate and the bottom of said second container.

5. The incense burner in accordance with claim 2, wherein said heating plate and the bottom part of said first container are both made of metal.

6. The incense burner in accordance with claim 2, wherein the bottom of said second container is made of ceramic, porcelain or steatite.

7. The incense burner in accordance with claim 3, wherein said second container has a handle part along its peripheral part, and at least one leg on its bottom part for providing a space between said top cover and the bottom of said second container, and is retained on said top cover with said leg.

8. The incense burner in accordance with claim 3, wherein said second container has a handle part along its peripheral part, and at least one leg on its bottom for providing a space between said top cover and the bottom of said second container, and said leg is provided in a direction of said handle part.

9. The incense burner in accordance with claim 1, wherein said heater is urged to a direction of said heating plate by a top cover which covers a peripheral part of the heating plate, a heater spring, and a heater cover on which one end of said heater spring rests, and further comprising: a first conduction plate provided between said heating plate and said heater; and a fixing plate provided on said heater cover and positioned by said top cover or said heater cover, wherein said first conduction plate and said heater are positioned by said fixing plate.

10. The incense burner in accordance with claim 9, further comprising a rubber ring provided between said heating plate and said top cover, wherein said rubber ring is covered by said top cover, thereby to provide a space between the heating plate-side tip end of said top cover and said heating plate.

11. The incense burner in accordance with claim 1, wherein said heater is urged to a direction of said heating plate by said top cover which covers a periphery of the heating plate, a heater spring and a heater cover on which one end of said heater spring rests, and wherein said heater cover has a bearing part for supporting said heating plate.

12. The incense burner in accordance with claim 11, further comprising: a rubber ring provided between said heating plate and said top cover which covers a periphery of said heating plate, thereby to provide a gap between the bearing part of the heater cover on which one end of said heater spring rests and said heating plate, wherein said gap does not exceed a compression margin of said rubber ring.

13. The incense burner in accordance with claim 9, further comprising a second conduction plate provided between said heater spring and said heater, wherein said second conduction plate is positioned by said fixing plate.

14. The incense burner in accordance with claim 9, wherein said heater cover is retained on said top cover by rotating it.

15. The incense burner in accordance with claim 1, further comprising an outer lid-guard which has a plurality of openings and is provided for detachably covering said container, wherein a lower end of said outer lid-guard extends downwardly beyond a surface of said heating plate.

16. An incense burner comprising:
a heater;
a heating plate;
a top cover having a vertically piercing opening which has an inner fringe part provided along a periphery of the opening for holding said heating plate so that it may not escape upwards, and a lateral part for holding said heating plate and said heater stored in the opening from a bottom and for regulating positions of these components directly or by the aids of other components so that they may not move in a lateral direction or rotate;
a heater spring for urging said heater to a direction of said heating plate; and
a heater cover fixed on an underside of said top cover for providing an approximately closed space defined between said top cover and said heater cover, which accommodates at least said heater spring, said heater and said heating plate, wherein said heater cover supports said heater spring upwards.

17. The incense burner in accordance with claim 16, further comprising a fixing plate, wherein
an area of said heater is smaller than an area of the heating plate
said fixing plate stores and positions said heater so that said heater may not move laterally or rotate, and
a peripheral part of said top cover positions said fixing plate so that it may not move laterally or rotate in the state of the fixing plate being stored in the opening of said top cover from a bottom.

18. The incense burner in accordance with claim 16, wherein said heater cover has a bearing part on its peripheral part being in contact with a lateral part of said top cover,
said bearing part is higher than a central part of said heater cover in which said heater is placed, and
at least a part of a peripheral part of said heating plate is sandwiched between said bearing part and said top cover directly or through a rubber ring.

19. An incense burner comprising: an upper frame unit comprising on its top a heating body which heats an aroma material; a light source; a pillar part made from transparent or translucent material, having a pillar encircling said light source; an outer frame made from transparent or translucent material encircling an outer side of said pillar part; and a lower frame unit for covering a bottom side opening of said outer frame, wherein
said outer frame is sandwiched between said upper frame unit and said lower frame unit, and
said lower frame unit is fixed to said pillar part with a fastening member.

20. The incense burner in accordance with claim 19, wherein said pillar part is attached to the upper frame unit by engagement prior to the fixing of the outer frame.

21. The incense burner in accordance with claim 20, wherein
said upper frame unit is configured with an upper frame and a heating body unit having the heating body, and
said pillar part is provided with a plurality of arm parts with attachment pieces on its top, and is engaged with said heating body unit by making said arm parts with attachment pieces pass through a plurality of fitting holes provided in said heating body unit and then rotating said pillar part.

22. The incense burner in accordance with claim 19, wherein said upper frame unit is configured with an upper frame and a heating body unit having the heating body, and
said pillar part is fixed to said upper frame after being engaged with said heating body unit, thereby making its detachment from said upper frame unit unable.

23. The incense burner in accordance with claim 21, wherein each of the arm-attachment pieces provided on the pillar part is divided into two parts, one of said divided arm-attachment pieces is made to be easily bent at the time of fixing the lower frame unit to said pillar part with a fastening member.

24. The incense burner in accordance with claim 21, wherein the arm-attachment pieces on the pillar part are provided on a position outside of the position where the heating body unit is disposed.

25. An incense burner comprising: an upper frame unit having a heating body for heating an aroma material; a pillar part; an outer frame disposed outside of said pillar part; and a lower frame unit, wherein
said pillar part is combined with said upper frame unit, and
said pillar part is combined with said lower frame unit in a state where said outer frame is sandwiched between a peripheral part of said upper frame unit and a peripheral part of said lower frame unit.

26. The incense burner in accordance with claim 25, wherein said pillar part is engaged with said upper frame unit, by making a plurality of arm-attachment pieces provided on the pillar part pass through fitting holes provided on the upper frame unit and then rotating the pillar part, and
at least a part of the plurality of the arm-attachment pieces bends in a state where said pillar part engages with said upper frame unit.

27. The incense burner in accordance with claim 19 further comprising a control part, wherein
said control part has a switch with a manipulation part which can control electric power supply to the heating body, and
said pillar part is attached to the lower frame unit having a slider sliding part which includes a slider for operating the manipulation part of said switch, an under frame provided with a first urging member for urging said slider, and an under frame cover.

28. The incense burner in accordance with claim 27, wherein said slider contains, therein, a push rod, and a second urging member for urging said push rod to a direction of the manipulation part of the switch,
a first urging member for urging the slider for operating the manipulation part of said switch is provided, and
said second urging member serves to moderate an outside force exerted on the manipulation part of the switch, after the switch manipulation part reaches a limit of its movable range.

29. An incense burner comprising:
a heating body for heating an aroma material;
a switch for controlling power supply to said heating body;
a housing having an opening on its bottom;
a slider partly stored in said housing for operating said switch directly or through a switch manipulation part and whose lower end projects downwards from said bottom opening; and
a first urging member for urging said slider downwards, wherein
in a state where the incense burner is placed on a plane, a lower end of said slider reaches a level of approximately the same as a bottom part of said housing and said switch permits power supply to said heating body and
in a state where the incense burner falls down, said slider projects more from said bottom opening by being urged by said first urging member, compared with a referred state where the incense burner is placed on a plane, and said switch shuts off power supply to said heating body.

30. The incense burner in accordance with claim 29, wherein
said slider comprises a slider main body; a push rod; a second urging member, one end of which is supported by said slider main body and the other end of which urges said push rod upwards,
said housing comprises a slider stopper for limiting upward movement of said slider main body,
said push rod and said second urging member are stored in said slider main body, and
in a state where said slider main body is pushed upward to hit said slider stopper, said push rod is pushed upward by said second urging member, operates said switch to be in a conductive state, while maintaining resiliency of said second urging member.

31. The incense burner in accordance with claim 19, wherein the control part is held by the pillar part and the lower frame unit.
